(19) United States
(12) Patent Application Publication
(10) Pub. No.: US 2022/0016197 A1
(43) Pub. Date: Jan. 20, 2022

(54) ZINGIBER ZERUMBET EXTRACT FOR COMBATING DANDRUFF

(71) Applicant: PIERRE FABRE DERMO-COSMETIQUE, BOULOGNE-BILLANCOURT (FR)

(72) Inventors: Joëlle LUC, FENOUILLET (FR); Isabelle ROUX, DONNEVILLE (FR); Christel FIORINI, TOULOUSE (FR); Philippe JOULIA, VILLENOUVELLE (FR); Alexandre PANEL, SAINT JUERY (FR)

(73) Assignee: PIERRE FABRE DERMO-COSMETIQUE, BOULOGNE-BILLANCOURT (FR)

(21) Appl. No.: 17/295,993

(22) PCT Filed: Dec. 4, 2019
(86) PCT No.: PCT/EP2019/083721

§ 371 Date: May 21, 2021

(30) Foreign Application Priority Data
Dec. 6, 2018 (FR) 1872434

(51) Int. Cl. A61K 36/9068; A61K 31/4412; A61K 31/555; A61P 17/08; A61P 31/10

(52) U.S. Cl. CPC A61K 36/9068; A61K 31/4412; A61P 31/10; A61P 17/08; A61K 31/555

(57) ABSTRACT

The present invention relates to the use of an extract of Zingiber zerumbet, in particular of Zingiber zerumbet rhizome, or of a dermatological or cosmetic composition comprising such an extract, for combating dandruff and/or preventing the formation thereof.

### TECHNICAL FIELD

The present invention relates to a Zingiber zerumbet extract and compositions containing said extract for their applications in the fields of cosmetics and dermatology for combating dandruff.

### PRIOR ART

Dandruff affects one out of every two people in France, its presence is benign, but it gives an unattractive appearance.

Dandruff is flakes of skin, and more precisely superficial scalp cells that have flaked off, but are unusually large, thicker, or more numerous than normal cells. The scalp, like all skin, normally renews itself by removing superficial cells from the epidermis. Dandruff is said to be dry or oily.

Dry dandruff is visible to the naked eye and appears as patches of the horny layer of the scalp. It is white, flat and comes off spontaneously. It is often accompanied by unpleasant itching. It is generally associated with a dry scalp.

Oily dandruff is rather small, round, yellowish and oily. It adheres to the scalp and hair. It forms a yellowish and sticky layer. It is accompanied by itching. In general, it is the consequence of seborrhea and thus often associated with an oily scalp and hair.

Dandruff is a disorder of epidermal formation. A healthy scalp has dead cells every day without it being noticed. This cell renewal occurs on a regular cycle of about 28 days. During this process, the epidermal cells have time to properly finalize their maturation, to gradually dissociate themselves from each other and, once they reach the surface, to detach themselves one by one invisibly. In the case of dandruff, the division of the cells of the basal layer of the epidermis is too rapid under the action of an inflammation of the scalp. Cell renewal then takes place in 5 to 14 days. The cells that rise to the skin's surface do not have time to lose their water and harden. They reach the surface without being sufficiently dried out, which forms clusters of cells on the scalp, which become visible: this is dandruff.

There are two dandruff states depending on the severity:
light and discreet dandruff: these are small whitish flakes corresponding to highly volatile clusters of dead cells that fall from the scalp and sprinkle onto clothes;
seborrheic dermatitis: this is dandruff associated with a strong inflammation of the scalp inducing redness and itching.

The epidermis of the scalp is covered with a lipidic film called sebum whose role is, inter alia, to protect it from drying out. Malassezia furfur (M. furfur), sometimes also called Pityrosporum ovale, is a yeast belonging to the group of Fungi imperfecta and is part of the commensal flora of humans. It is present in practically 100% of the population. It is established that this fungus is a low-pathogenic agent to humans. However, it is also established that Malassezia furfur, a lipophilic yeast, is responsible in about 3% of the population for dermatoses such as adult seborrheic dermatitis. This fungus grows easily in oily environments, such as the scalp, where sebum is secreted in particularly high amounts, thus creating a favorable environment for its development. This fungus can “digest” lipids present in the sebum and produce fatty acids that are particularly irritating to the skin and scalp. Produced in too large amounts, they can induce an inflammation that causes dandruff. For about 50% of individuals, sebum and Malassezia furfur coexist in good harmony. For the remaining 50%, an imbalance may occur, with several factors being taken into account. Not everyone secretes the same amount of sebum. Some people secrete a sufficiently large amount to be conducive to the excessive development of Malassezia furfur and thus to the formation of dandruff. Furthermore, the amount of sebum varies over time, in particular according to hormonal secretions. Finally, other factors can promote the appearance of dandruff, such as stress, fatigue, pollution, lack of sunlight, certain illnesses such as Parkinson's disease, certain medications such as neuroleptics, etc.

Dandruff often appears in adolescence at the time of puberty when the secretion of sebum begins. Men seem to be affected more frequently than women, undoubtedly under the effect of androgen hormones.

Anti-dandruff compositions, particularly in the form of shampoos, are known and have been commercially available for many years. They generally contain chemical active agents such as ketoconazole, zinc pyrithione, octopirox, piroctone olamine, salicylic acid, selenium sulfide and azelaic acid. Certain compounds, in particular zinc pyrithione, are suspected of inducing environmental problems, particularly ecotoxicity by inducing toxicity in fish. It is therefore important to find new active agents that are natural and environmentally friendly, while being as effective as chemical active agents.

Thus, despite the many options currently available, consumers still need new products to combat dandruff and/or prevent its formation.

### SUMMARY OF THE INVENTION

Surprisingly, the inventors discovered that Zingiber zerumbet extracts had activity on Malassezia furfur at least of the same order as zinc pyrithione in the model used.

The purpose of the present invention is thus to propose an effective treatment against dandruff. Preferably, this treatment is carried out topically, which allows a better effectiveness.

The object of the present invention is thus a Zingiber zerumbet extract for use, preferably for topical use, in the prevention and/or treatment of dandruff.

The present invention also has as its object the use, preferably the topical use, of a Zingiber zerumbet extract in the prevention and/or treatment of dandruff.

The present invention also has as its object the use of a Zingiber zerumbet extract for the manufacture of a dermatological or cosmetic composition, preferably a topical dermatological or cosmetic composition, intended for the prevention and/or treatment of dandruff.

The present invention also has as its object a method for the prevention and/or treatment of dandruff comprising the administration, in particular by topical application, of an effective amount of a Zingiber zerumbet extract to a person in need thereof.

The present invention is also aimed at a cosmetic method for the treatment and/or prevention of dandruff comprising the administration, in particular by topical application, of a Zingiber zerumbet extract.

### DETAILED DESCRIPTION

### Zingiber Zerumbet

Zingiber zerumbet is an herbaceous plant of the genus Zingiber of the family Zingiberaceae native to Asia. Its common names are bitter ginger, zerumbet or ginger shampoo. It is a plant native to India, which gradually spread westward to Madagascar, to East Asia (China, Thailand, Cambodia), then to Polynesia and the Hawaiian Islands with the first Polynesian settlers. It also exists in tropical America, for example in Guyana and Martinique.

Zingiber zerumbet is a tubular perennial plant that is found naturally in damp and shady places. It is often found near rivers, waterfalls, and other water sources. This asexual species reproduces by multiplying rhizome fragments. This plant is characterized by the presence of stems 1 to 2 m high. The leaves and inflorescences grow from a thick rhizome or an underground stem. The leaves are thin and measure approximately 25-35 cm in length. The leaves are green and alternate. The leaf sheaths wrap around the stems and the leaf blades are oblanceolate or narrowly oblong in shape. The flower bracts are green when young and turn red with age. The flowers themselves emerge from between the bracts and are inconspicuous, tri-petalled and pale yellow in color. The fruit is white, hairless, thin-walled and about 1.5 cm in length. The seeds are ellipsoidal and black. The rhizome is perennial, thick, aromatic, and yellow.

The rhizomes of Zingiber zerumbet contain compounds of various chemical classes such as terpenes, polyphenols, and sugars. The essential oil is rich in terpenes such as zerumbone. The polyphenols are of two types:
flavonoids such as kaempferol and quercetin derivatives, and,
other phenolic compounds.

The rhizome is traditionally used orally or locally. It is used to treat indigestion and pain, toothache, and sprains. Orally, the roots are used in food as a condiment as well as for their benefits to treat headaches, sore throats, and rheumatism. Zingiber zerumbet extract inhibits cyclooxygenase (COX) and the synthesis of inflammatory mediators in both the central and peripheral nervous system.

### Extract According to the Invention

“Zingiber zerumbet extract” is intended to mean the extraction product of all or part of the Zingiber zerumbet plant, preferably its rhizomes.

“Extraction product” means the product obtained after extraction of the Zingiber zerumbet plant, and in particular of the rhizomes of Zingiber zerumbet, with a solvent, called extraction solvent, (i.e., a liquid solution in the extraction solvent) optionally in concentrated or dry form after partial or total evaporation of the extraction solvent. It can thus be a dry extract.

In the sense of the present invention, “dry extract” means an extract free of extraction solvent or containing only insignificant trace amounts thereof. Such a dry extract thus contains only material derived from Zingiber zerumbet, in particular its rhizomes. It may also contain insignificant traces of extraction solvent.

In the context of the present invention, the Zingiber zerumbet extract is advantageously obtained from the rhizomes of the plant.

Zingiber zerumbet, especially its rhizome, can be fresh, dry, whole, cut or ground and then subjected to an extraction step.

A process for the preparation of an extract according to the invention comprises a step of extraction of Zingiber zerumbet, in particular of its rhizome, by an extraction solvent. The extraction may be carried out at room temperature (e.g., between 15 and 30° C., in particular between 20 and 25° C.) and/or at atmospheric pressure or at high pressure.

The extraction solvent can be a solvent or a mixture of solvents selected from:
water
alcohols such as ethanol, methanol, isopropanol
ketones including acetone and methyl ethyl acetone
ethyl acetate
methylene chloride
diisopropyl ether
hexane, heptane,
CO₂ and more particularly supercritical CO₂.

Extraction can be carried out by different technologies, in particular so-called “green” technologies, such as at atmospheric pressure (using ultrasound, microwaves, leaching, maceration, decoction . . . ) or at high pressures (e.g., 10 to 150 bars), with extraction solvents in a super- or sub-critical state (such as CO₂ or water), and/or at temperatures comprised between 20° C. to 250° C.

Advantageously, the Zingiber zerumbet extract, in particular an extract of its rhizomes, will be composed primarily of a terpenic fraction and possibly a phenolic fraction. The extract according to the invention may also comprise a saccharide fraction.

“Terpenic fraction” means a fraction of the extract containing lipophilic terpenic (monoterpenic, sesquiterpenic, diterpenic or triterpenic) compounds. It advantageously represents from 10 to 100% w/w of the dry extract.

“Phenolic fraction” means a fraction of the extract containing compounds having 1 or more phenol groups such as flavonoids, predominantly glycosylated, mainly derived from kaempferol such as kaempferol, 3-O-(kaempferol) 2,4-diO-acetylrhamnoside, kaempferol 4-O-acetylrhamnoside, kaempferol 3-O-rhamnoside and, to a lesser extent, dihydrokaempferol.

It represents advantageously from 0 to 70% w/w, in particular from 30 to 70% w/w of the dry extract.

“Saccharide fraction” means a fraction of the extract containing monosaccharides or disaccharides such as fructose, glucose, and sucrose. It advantageously represents from 0 to 30% w/w, in particular from 5 to 15% w/w of the dry extract.

According to a first embodiment, the extract according to the invention is a heptanic extract, i.e., the extraction solvent used is heptane.

According to a second embodiment, the extract according to the invention is an ethanolic extract, i.e., the extraction solvent used is ethanol.

According to a third embodiment, the extract according to the invention is obtained by extraction with supercritical CO₂, with or without co-solvent.

### Dermatological and Cosmetic Application

Surprisingly, the inventors discovered that a Zingiber zerumbet extract, in particular a Zingiber zerumbet rhizome extract, was endowed with anti Malassezia activity (Example 1).

The Zingiber zerumbet extract according to the invention, such as a Zingiber zerumbet rhizome extract, is thus useful for combating dandruff and/or preventing the formation of dandruff, in particular by topical application.

“Topical application/use” means application/use on the skin, in particular the scalp, mucous membranes and/or skin appendages, preferably on the scalp.

“Skin appendage” means head hair, body hair and/or nails, preferably head hair.

The term “combat dandruff” or “treat dandruff” means to decrease the number of dandruff and/or inhibit the development of dandruff.

The term “prevention” or “to prevent” the formation of dandruff means to decrease, delay and/or avoid the appearance of dandruff.

The Zingiber zerumbet extract according to the invention can be administered, in particular by topical application, once a month, once a week, twice a week, every other day, daily, or even twice a day.

### Dermatological or Cosmetic Composition

The present invention also relates to a dermatological or cosmetic (e.g., dermo-cosmetic) composition for use for combating dandruff and/or preventing the formation of dandruff, comprising a Zingiber zerumbet extract according to the invention, as defined above, and in particular a Zingiber zerumbet rhizome extract, with a dermatologically or cosmetically acceptable excipient, more particularly acceptable for topical application.

The present invention also has as its object the use, preferably the topical use, of a dermatological or cosmetic (e.g., dermo-cosmetic) composition comprising a Zingiber zerumbet extract according to the invention, as defined above, and in particular a Zingiber zerumbet rhizome extract, with a dermatologically or cosmetically acceptable excipient, more particularly acceptable for topical application, in the prevention and/or treatment of dandruff.

The present invention also has as its object a method for the prevention and/or treatment of dandruff comprising the administration, in particular by topical application, of an effective amount of a dermatological or cosmetic (e.g., dermo-cosmetic) composition comprising a Zingiber zerumbet extract according to the invention, as defined above, and in particular a Zingiber zerumbet rhizome extract, with a dermatologically or cosmetically acceptable excipient, more particularly acceptable for topical application, to a person in need thereof.

The present invention is also aimed at a cosmetic method for the treatment and/or prevention of dandruff comprising the administration, in particular by topical application, of a dermatological or cosmetic (e.g., dermo-cosmetic) composition comprising a Zingiber zerumbet extract according to the invention, as defined above, and in particular a Zingiber zerumbet rhizome extract, with a dermatologically or cosmetically acceptable excipient, more particularly acceptable for topical application.

In the present invention, “dermatologically or cosmetically acceptable” means that which is useful in the preparation of a dermatological or cosmetic composition, which is generally safe, non-toxic and neither biologically nor otherwise undesirable and which is acceptable for therapeutic or cosmetic use, in particular by topical application.

The extract according to the invention may be introduced into the composition according to the invention in the form of a dry extract or in the form of a solution in its extraction solvent (optionally after partial evaporation of the solvent to give a concentrated extract) or in another solvent. Indeed, the extraction solvent may be evaporated (completely or partially, preferably completely) and the dry or concentrated extract obtained, preferably the dry extract, may then be solubilized by another solvent to be in liquid form and thus constitute a fluid extract. This recovery solvent can be for example a C3-C5 glycol such as propylene glycol, butylene glycol, propane-1,3-diol or pentanediol, or glycerin.

In a particular embodiment, the dermatological or cosmetic compositions according to the invention comprise at least one other anti-dandruff active principle such as an antifungal like piroctone olamine, zinc pyrithione, ciclopirox or a mixture thereof.

According to another embodiment, the dermatological or cosmetic compositions according to the invention further comprise a celery seed extract, having clinically proven activity as an anti-dandruff agent.

The invention is preferably aimed at dermatological or cosmetic compositions in a form appropriate and suitable for topical application.

The dermatological or cosmetic compositions according to the invention may thus be in the forms which are usually known for topical administration, i.e., in particular lotions, shampoos, conditioners, balms, foams, gels, dispersions, emulsions, sprays, serums, masks or creams.

Advantageously, the compositions according to the invention may be in forms that are usually known for topical administration to the hair and scalp, i.e., particularly a shampoo, a conditioner, a hair cream, a hair lotion, a mask, or a spray, in particular a leave-in spray.

A distinction is thus made between formulated products that can be rinsed and formulated products that do not require rinsing.

These compositions generally contain, in addition to the Zingiber zerumbet extract according to the present invention, a physiologically acceptable medium, generally based on water or solvent, for example alcohols, ethers or glycols. They may also contain surfactants, complexing agents, preservatives, stabilizing agents, emulsifiers, thickeners, gelling agents, humectants, emollients, trace elements, essential oils, fragrances, dyes, moisturizing agents, or geothermal waters, etc.

Advantageously, the compositions according to the present invention will comprise 0.05 to 2%, preferably 0.1 to 1% by weight, even more preferably 0.1 to 0.8% of Zingiber zerumbet extract by weight of dry extract with respect to the total weight of the composition. In a preferred manner, the compositions according to the present invention will comprise 0.2% by weight of Zingiber zerumbet dry extract with respect to the total weight of the composition. In an equally preferred manner, the compositions according to the present invention will comprise 0.5% by weight of Zingiber zerumbet dry extract with respect to the total weight of the composition.

Such compositions can be manufactured according to processes well known to the person skilled in the art.

The following examples illustrate the invention without limiting its scope.

### EXAMPLES

### Example 1: Evaluation of the Anti Malassezia Activity of Different Zingiber zerumbet Extracts

Generally, anti-dandruff active agents are evaluated by their ability to inhibit the growth of M. furfur colonies (determination of Minimum Inhibitory Concentrations (MIC)) which is insufficient to predict lethal activity of M. furfur and therefore optimal removal of dandruff. Indeed, M. furfur may proliferate anew if it is not completely or sufficiently removed. Consequently, the effectiveness of an anti-dandruff active agent must also be shown by demonstrating its effect on the lethality of M. furfur. This component is generally measured by estimating the decay of microbial colonies over time.

In the context of this work, the model was optimized in terms of sample preparation, contact time and sample evaluation concentration.

The purpose of the test described below is to evaluate in a relevant way the anti Malassezia activity of different Zingiber zerumbet rhizome extracts and to compare it with that of pure zerumbone and with zinc pyrithione.

### Methods

Five formulas are tested in this test:
A compound used as reference: zinc pyrithione at 1.0% w/w in a mixture A comprising: sodium laureth sulfate 14.3%, alkyl dimethyl betaine 10.0%, decyl glucoside 3.8%, sorbitan monolaurate 2.0%, citric acid monohydrate 10% solution, qs pH=5; water qs 100;
A Zingiber zerumbet rhizome extract obtained by heptanic extraction from the dry and ground rhizome, at 2.0% w/w in mixture A;
A Zingiber zerumbet rhizome extract obtained by ethanolic extraction, at 2.0% w/w in mixture A;
A Zingiber zerumbet rhizome extract obtained by supercritical CO₂ in a water-soluble excipient (butylene glycol) at 3.0% w/w in mixture A. This is a commercial material called Legance®, sold by the firm Sederma. The INCI name of this material is: Butylene glycol-Zingiber Zerumbet Extract.

The procedure for this test is as follows:

An inoculation of 20 g of test product is carried out with 1% inoculum of Malassezia furfur titrating at 10⁷ CFU/ml. After a determined contact time, 1 g of product is taken and a 1/10 dilution is carried out in the neutralizing diluent. Successive dilutions at 1/10 up to 1/1000 dilution are carried out in the diluent. 1 ml is filtered on a 0.45 μm membrane in ester and cellulose acetate in the presence of 50 ml of sterile demineralized water (SDW), then two rinses are carried out with 50 ml of SDW.

The chosen contact times are 1 h, 2 h, 5 h and 8 hours. The neutralizing diluent is TSB+10% polysorbate 80, the diluent is tryptone-salt. The rinsing liquid is sterile demineralized water. The strain and culture conditions are summarized in Table 1 below:

TABLE 1

| | |
|---|---|
| Strain | Malassezia furfur CBS1878 |
| Culture medium | DIXON agar |
| Incubation conditions | 4 to 6 days at 30-35° C. |

### Results

The results obtained with these different extracts are presented in Table 2 below:

TABLE 2

| | | Enumeration of residual microorganisms after contact | | | |
|---|---|---|---|---|---|
| | Number of | (CFU/g, log reduction value) | | | |
| Products | μorganisms | Contact time | | | |
| tested | CFU/g | 1 hour | 2 hours | 5 hours | 8 hours |
| Compound 1 | 1.2 × 10⁵ | 7.2 × 10⁴ | 4.8 × 10⁴ | 4.9 × 10³ | <1.0 × 10² |
| | | 0.23 | 0.40 | 1.39 | >3.08 |
| Compound 2 | 5.5 × 10⁴ | 3.0 × 10² | 1.0 × 10¹ | <1.0 × 10¹ | <1.0 × 10¹ |
| | | 2.26 | 3.74 | >3.74 | >3.74 |
| Compound 3 | 5.5 × 10⁴ | 1.5 × 10⁴ | 7.2 × 10² | <1.0 × 10¹ | <1.0 × 10¹ |
| | | 0.56 | 1.88 | >3.74 | >3.74 |
| Compound 4 | 1.2 × 10⁵ | 6.2 × 10⁴ | 3.3 × 10⁴ | 3.1 × 10³ | 1.3 × 10² |
| | | 0.29 | 0.56 | 1.59 | 2.97 |

Compound 1: zinc pyrithione 1.0%.
Compound 2: heptanic extract of Zingiber zerumbet, 2.0%.
Compound 3: ethanolic extract of Zingiber zerumbet, 2.0%.
Compound 4: supercritical CO₂ extract of Zingiber zerumbet, 3.0%.

Zinc pyrithione (compound 1) shows activity on Malassezia furfur from 1 hour of contact. At 8 hours of contact, the reduction in the number of microorganisms exceeds 3 log. These results validate this test.

The different Zingiber zerumbet rhizome extracts show an anti Malassezia activity at least similar to that of the reference product. The inventors thus demonstrate that these extracts have a very advantageous anti-dandruff activity.

1-13. (canceled)

14. A method for preventing and/or treating dandruff comprising the administration of an effective amount of a Zingiber zerumbet extract to a person in need thereof,
wherein the Zingiber zerumbet extract is a Zingiber zerumbet extract obtained by extraction with supercritical CO₂ or a heptanic Zingiber zerumbet extract.

15. The method according to claim 14, wherein the Zingiber zerumbet extract is a Zingiber zerumbet extract obtained by extraction with supercritical CO₂.

16. The method according to claim 14, wherein the Zingiber zerumbet extract is a heptanic Zingiber zerumbet extract.

17. The method according to claim 14, wherein the Zingiber zerumbet extract is a Zingiber zerumbet rhizome extract.

18. The method according to claim 14, wherein the administration of the Zingiber zerumbet extract is a topical application.

19. A method for preventing and/or treating dandruff comprising the administration of an effective amount of a dermatological or cosmetic composition comprising at least one Zingiber zerumbet extract with at least one dermatologically or cosmetically acceptable excipient,
wherein the Zingiber zerumbet extract is a Zingiber zerumbet extract obtained by extraction with supercritical CO₂ or a heptanic Zingiber zerumbet extract.

20. The method according to claim 19, wherein the Zingiber zerumbet extract is a Zingiber zerumbet extract obtained by extraction with supercritical CO₂.

21. The method according to claim 19, wherein the Zingiber zerumbet extract is a heptanic Zingiber zerumbet extract.

22. The method according to claim 19, wherein the Zingiber zerumbet extract is a Zingiber zerumbet rhizome extract.

23. The method according to claim 19, wherein the dermatological or cosmetic composition comprises 0.05 to 2% of the Zingiber zerumbet extract by weight of dry extract with respect to the total weight of the dermatological or cosmetic composition.

24. The method according to claim 23, wherein the dermatological or cosmetic composition comprises 0.1 to 1% of the Zingiber zerumbet extract by weight of dry extract with respect to the total weight of the dermatological or cosmetic composition.

25. The method according to claim 24, wherein the dermatological or cosmetic composition comprises 0.2 to 0.8% of the Zingiber zerumbet extract by weight of dry extract with respect to the total weight of the dermatological or cosmetic composition.

26. The method according to claim 19, wherein the administration of the dermatological or cosmetic composition is a topical application.

27. The method according to claim 19, wherein the dermatological or cosmetic composition further comprises at least one other anti-dandruff active principle.

28. The method according to claim 27, wherein the at least one other anti-dandruff active principle is an antifungal.

29. The method according to claim 28, wherein the antifungal is piroctone olamine, zinc pyrithione, ciclopirox or a mixture thereof.

30. The method according to claim 19, wherein the dermatological or cosmetic composition further comprises a celery seed extract.
